(19) United States
(12) Patent Application Publication
(10) Pub. No.: US 2026/0338901 A1
(43) Pub. Date: Oct. 29, 2026

(54) Systems and Methods for Free Breathing Cardiac Cine Imaging at 3T with Variable Flip Angle and Regional Virtual Coils

(71) Applicant: University of Virginia Patent Foundation, Charlottesville, VA (US)

(72) Inventors: Kang Yan, Charlottesville, VA (US); Quan Dou, Charlottesville, VA (US); Zhixing Wang, Charlottesville, VA (US); Xue Feng, Ashburn, VA (US); Craig H. Meyer, Charlottesville, VA (US)

(21) Appl. No.: 19/661,662

(22) Filed: Apr. 28, 2026

Related U.S. Application Data
(60) Provisional application No. 63/796,251, filed on Apr. 28, 2025.

(51) Int. Cl. G01R 33/561; A61B 5/00; A61B 5/055; G01R 33/48; G01R 33/56

(52) U.S. Cl. CPC G01R 33/5614; A61B 5/0044; A61B 5/055; A61B 5/7207; G01R 33/4824; G01R 33/5608

(57) ABSTRACT

Variable flip angle sequences for balanced steady state free precession magnetic resonance imaging can determined by modifying the extended-phase-graph (EPG)-based optimized flip angle scheme to simulate flip angles within a single measurement (a single measurement consists of fully sampled spiral interleaves). The resulting simulated flip angle curve closely resembles a sine wave. The model allows for using extend phase graphs in spiral in-out sampling protocols for MR imaging. Also, region-optimized virtual coils algorithms are used for computational efficiency.

### CROSS-REFERENCE TO RELATED APPLICATIONS

This application claims priority to and the benefit of U.S. provisional patent application No. 63/796,251, filed on Apr. 28, 2025, and titled “Method and System for Optimization of Free-breathing Spiral Cardiac Cine Imaging at 3T with Variable Flip Angle Scheme and Region-Optimized Virtual Coils (ROVir),” the disclosure of which is hereby incorporated by reference herein in its entirety.

### STATEMENT OF GOVERNMENT RIGHTS

This invention was made with government support under grant number EB028773 awarded by the National Institutes of Health. The government has certain rights in the invention.

### BACKGROUND

Cardiac magnetic resonance (CMR) cine imaging is widely regarded as the gold-standard technique for the non-invasive assessment of cardiac function. Typically, images are acquired using a breath-held 2D segmented ECG-gated balanced steady-state free precession (bSSFP) pulse sequence. Prospective or retrospective ECG-gating is used to synchronize segmented data acquisition to the cardiac cycle over multiple heartbeats during a breath-hold to generate images at multiple cardiac phases across the cardiac cycle.

The approach of using ECG triggering and breath-hold acquisition has several limitations. Firstly, the ECG signal can be distorted due to the magnetohydrodynamic effect [1], rapid switch of magnetic gradients [2], as well as radiofrequency interference [3,4] resulting in mis-triggering. This distortion is worse at higher field strengths such as 3T. Furthermore, placement of the ECG leads requires expertise and increases the time to prepare the patient for the CMR exam. Additionally, a significant number of patients are not able to adequately hold their breath during cine acquisition, resulting in motion artifacts and the need to repeat image acquisition of the same slice location on subsequent breath-holds. Even if the patient can perform good breath-holds, this approach is inefficient as it requires 10-12 breath-holds to cover the left ventricle (LV) and requires coordination between the operator and the patient.

To partially alleviate issues caused by cardiac and respiratory motion, the current solutions can be separated into 3 categories: navigator-based methods, real-time methods, and self-gating methods. Navigator-echo based methods which accept or reject data based on the position of the diaphragm have been used to account for respiratory motion [5-8]. However, the usage of diaphragmatic navigators typically precludes retrospective ECG gating, and the total scanning time is prolonged depending on respiratory gating efficiency. More recently, projection navigators acquired during SSFP have been successfully utilized to perform respiratory tracking without the need for a separate diaphragmatic navigator [9], but this approach is still limited by navigator gated efficiency. These methods account for respiratory motion, but do not provide a mechanism to account for cardiac motion.

Other “self-navigated” techniques [10,11] have been proposed to eliminate the need for ECG synchronization by acquiring and processing additional MR signals to derive cardiac cycle timing information. Relative to ECG-gated techniques, methods that use extra lines of data to acquire the self-gating signals result in decreased imaging efficiency. In the clinical setting, when the ECG or breath-holds do not perform adequately, operators may resort to real-time imaging techniques. Although these real-time methods do not require ECG gating, they have been shown to sacrifice spatial and/or temporal resolution [12-14].

Thus, there is a growing interest in self-gated free-breathing approaches that do not compromise achievable resolution. While applying ECG triggering, some studies [15-20] relied on the acquired data itself to derive respiratory signals, such as using filtered data or projection data. The acquired dataset were usually separated into different respiratory states [18,21,22] and reconstruction was performed using data from a subset of the respiratory states, or by using motion correction to combine data from different respiratory states. Other studies extracted the cardiac motion from the acquired data during breath-holds [23-25] or free-breathing [21,22,26]. Previously reported cardiac self-gating approaches have been shown to use the k-space center point [11,27] or center k-space line [12,15,16,23] as navigator signals. Some techniques [23,28] also used image-based methods to obtain self-gating signals.

In terms of sampling strategy, most studies have utilized Cartesian and radial trajectories [10,11,15-17,23,28]. A recent study has used a breath-held cardiac self-gated spiral technique to quantify coronary artery vasodilation [25]. To date, no studies have explored the use of free-breathing cardiac and respiratory self-gated golden angle spiral trajectories for the evaluation of cardiac anatomy and function.

Despite a number of recent advances, there are still several limitations to be overcome. Firstly, these approaches usually require careful selection of receive coil elements to obtain self-gating signals as each coil has different sensitivity to cardiac motion and respiratory motion. Secondly, it is inefficient to discard data acquired at undesired respiratory phases. Finally, for imaging at 3T, bSSFP sequences typically require frequency-scouts and careful shimming to avoid off-resonance artifacts such as banding artifact and may be less robust for automatic free breathing acquisition.

It is with respect to these and other considerations that the various aspects of the present disclosure as described below are presented.

### SUMMARY

Other aspects and features according to the example embodiments of the disclosed technology will become apparent to those of ordinary skill in the art, upon reviewing the following disclosure.

In one implementation, a computer implemented method of specifying a magnetic resonance (MR) imaging protocol to provide output images with high contrast sections and reduced artifacts includes using a computer having a processor in data communication with computer memory storing software that implements steps to establish a selected variable flip angle pulse sequence that initiates, within a target field of view, corresponding magnetization states that are sampled with a corresponding spiral in-out trajectory for MR imaging; and establish a protocol for reconstructing image data from sampled magnetization states, wherein the protocol utilizes an automated virtual coil selection that manages a signal to interference ratio in the output images. The variable flip angle pulse sequence is determined by using the software to perform computerized modeling steps that produce respective extended phase graphs for MR test data, the modeling steps comprising identifying a target k-space distribution suitable for providing the output images with the corresponding spiral in-out sampling trajectory; using the respective extended phase graphs to assess variable flip angle sequences applied to the test MR image data and sampled with at least one spiral in-out sampling trajectory; saving a selected variable flip angle sequence that provides a resulting k-space distribution, upon spiral in-out sampling, that approaches the target k-space distribution, and wherein the software further applies the automated virtual coil selection by selecting a range of eigenvalues for inclusion in reconstruction of the output images derived from the resulting k-space distribution.

### BRIEF DESCRIPTION OF THE DRAWINGS

Reference will now be made to the accompanying drawings, which are not necessarily drawn to scale. The patent or application file contains at least one drawing executed in color. Copies of this patent or patent application publication with color drawing(s) will be provided by the Office upon request and payment of the necessary fee.

FIG. 1 is a system diagram illustrating an imaging system capable of implementing aspects of the present disclosure in accordance with one or more embodiments.

FIG. 2 is a computer architecture diagram showing a computing system capable of implementing aspects of the present disclosure in accordance with one or more embodiments.

FIG. 3 is a flow diagram showing operations of a method for performing Free-breathing Spiral Cardiac Cine Imaging at 3T with Variable Flip Angle Scheme and Region-Optimized Virtual Coils (ROVir) in accordance with an embodiment of the present disclosure.

FIG. 4 is a series of images representing steps of the method of this disclosure.

FIG. 5 is a series of images representing the region-optimized virtual coil algorithm steps of this disclosure.

FIG. 6 is a comparison of reconstructed images at an under-sampling rate of R=8 from CFA and CFA acquisitions in basal and mid slices.

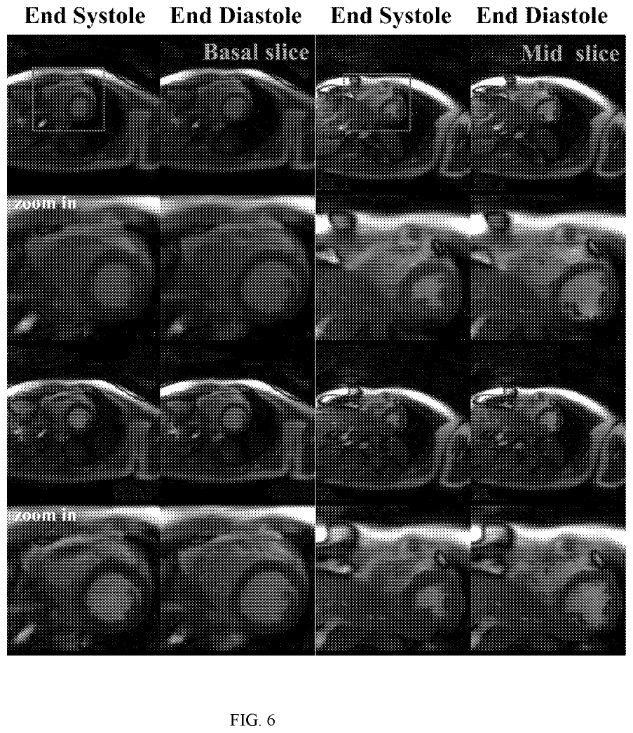

FIG. 7 is a quantitative analysis of CFA and VFA acquisitions in terms of CNR within one heartbeat for basal and mid slices.

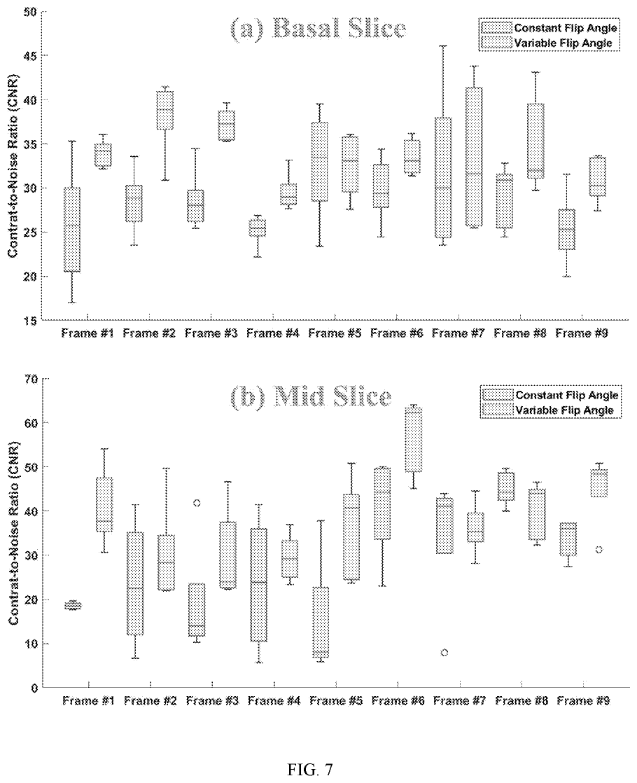

FIG. 8 is a reconstructed free-breathing spiral cardiac cines from VFA acquisition using two different coil compression methods.

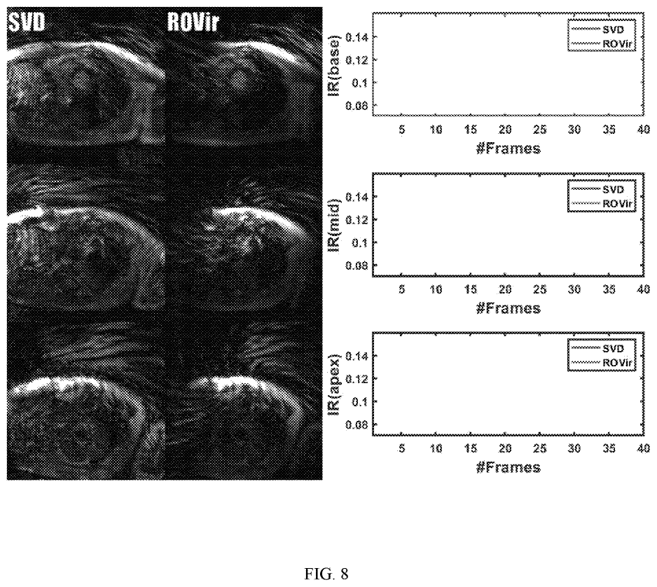

FIG. 9 is an example of extended phase graphs used in this disclosure.

### DETAILED DESCRIPTION

Cardiac MRI is an imaging technique for the detection and monitoring of cardiac diseases in clinical settings. Recent studies [B1, B2] have demonstrated that a spiral in/out bSSFP pulse sequence can be a viable alternative for fast, free-breathing cardiac cine imaging at 1.5 Tesla (1.5T). However, when imaging at higher fields to achieve improved SNR, the SAR increases quadratically. Given SAR limitations, lower flip angles must be used, resulting in reduced image contrast. Additionally, swirl artifacts, similar to the streak artifacts seen in radial cardiac cine imaging, emerge from the edges of the FOV due to field inhomogeneity, gradient infidelity, or insufficient fat suppression, further degrading image quality.

To overcome these issues, the present disclosure developed, among other things, an optimized variable flip angle (VFA) scheme to enhance image contrast without increasing SAR. The present disclosure also introduced, among other things, a region-optimized virtual coils (ROVir) algorithm to mitigate image artifacts.

In some embodiments, this disclosure makes us of extended phase graphs (EPGs) to select a variable flip angle pulse sequence that initiates, within a target field of view, corresponding magnetization states that are sampled with a corresponding in-out trajectory for MR imaging. Reference [B3] cited below discusses one method of creating extended phase graphs and using those graphs in determining diffusion weighted SPLICE (split acquisition of fast spin-echo signals). Reference [B3] uses extended phase graphs (EPGs) to determine the best RF pulse sequence to establish a targeted point spread function (PSF). The target of Reference [B3] optimizes the flip angles for a linear k-space sampling order.

This disclosure has determined how to establish a variable flip angle RF pulse sequence that is more useful for spiral in-out trajectory sampling in k-space.

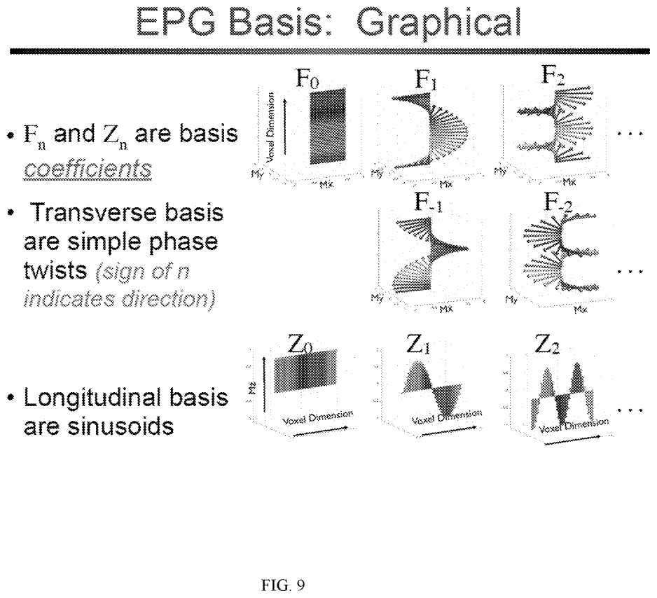

The basic notions of extended phase graphs (EPGs) are succinctly shown in Ref. [B8]: Brian Hargreaves and Karla Miller ISMRM 2013: Educational E-Poster #3718 and a corresponding slide set used to teach Stanford University's course Radiology 229 (selection shown in FIG. 9. As explained by Hargreaves, in EPG analysis, gradient areas are quantized into units that give a phase twist of 2pi across a voxel. Then, EPG analysis allows for representing large groups of spins with Fourier basis and accurately calculate magnetic resonance MR signals. EPG analysis allows for common representations for magnetization in vectors Mx, My, Mz. In common notation, the spins in a voxel are based on the location z across the voxel. Mxy(z) and Mxy*(z) are transverse magnetization entries in the magnetization array. Mz(z) is longitudinal magnetization. Extended Phase Graphs represent a large number of spins using a compact basis set of Fn and Zn coefficients. Computer programs, such as that discussed in Reference [B3], can iteratively estimate the coefficients to estimate MR data that would result from corresponding RF VFA sequences. As noted in [B3] a target output can be identified to supervise the iterative process to converge upon a variable flip angle (VFA) sequence that can approximate the target output.

This disclosure allows for using EPGs in the context of spiral readouts instead of linear k-space analysis by adjusting the matrices and groups of magnetization states to reflect spiral sampling.

In some aspects, the disclosed technology relates to systems, methods, and computer-readable medium for magnetic resonance based imaging. Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, referred to herein as an “area of interest” or a “region of interest.”

A detailed description of aspects of the disclosed technology, in accordance with various example embodiments, will now be provided with reference to the accompanying drawings. The drawings form a part hereof and show, by way of illustration, specific embodiments and examples. In referring to the drawings, like numerals represent like elements throughout the several figures.

Embodiments of the present disclosure include MRI-based imaging techniques. The present disclosure sets forth to enhance image quality of free-breathing spiral cardiac cine imaging at 3 Tesla (3T), with a particular focus on improving image contrast and reducing artifacts.

Example, Non-limiting Goals: This study aims to, among other things, demonstrate that optimizing both data acquisition and the reconstruction algorithm can enhance overall image quality by improving image contrast and reducing artifacts.

Example, Non-limiting Approach: A variable flip angle scheme based on extended phase graphs CEPG) simulation and a region-optimized virtual coils algorithm were developed.

Example, Non-limiting Results: The in vivo experiments demonstrate that, among other things, the proposed methods outperform the conventional acquisition and reconstruction methods at 8-fold under-sampling rate.

The present disclosure submits that reduced image contrast, along with interference artifacts can restrict the clinical use of free-breathing spiral cardiac cine imaging at 3T. The optimizations presented here offer significant potential to, among other things, broaden the clinical applicability of this technique at 3T.

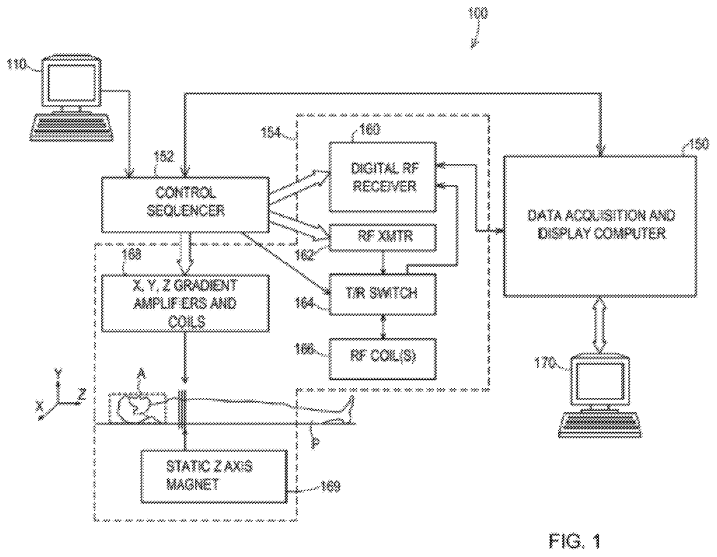

FIG. 1 is a system diagram illustrating an imaging system capable of implementing aspects of the present disclosure in accordance with one or more example embodiments. FIG. 1 illustrates an example of a magnetic resonance imaging (MRI) system 100, including a data acquisition and display computer 150 coupled to an operator console 110, an MRI real-time control sequencer 152, and an MRI subsystem 154. The MRI subsystem 154 may include XYZ magnetic gradient coils and associated amplifiers 168, a static Z-axis magnet 169, a digital RF transmitter 162, a digital RF receiver 160, a transmit/receive switch 164, and RF coil(s) 166. The MRI subsystem 154 may be controlled in real time by control sequencer 152 to generate magnetic and radio frequency fields that stimulate magnetic resonance phenomena in a subject P to be imaged, for example to implement magnetic resonance imaging sequences in accordance with various embodiments of the present disclosure. Reconstructed images, such as contrast-enhanced image(s) of an area of interest A of the subject P may be shown on display 170.

The area of interest A shown in the example embodiment of FIG. 1 corresponds to a head region of subject P, but it should be appreciated that the area of interest for purposes of implementing various aspects of the disclosure presented herein is not limited to the head area. It should be recognized and appreciated that the area of interest in various embodiments may encompass various areas of subject P associated with various physiological characteristics, such as, but not limited to the head and brain region, chest region, heart region, abdomen, upper or lower extremities, or other organs or tissues. Various aspects of the present disclosure are described herein as being implemented on portions of the skeletal system of human subjects, for example cortical bone tissue.

It should be appreciated that any number and type of computer-based medical imaging systems or components, including various types of commercially available medical imaging systems and components, may be used to practice certain aspects of the present disclosure. Systems as described herein with respect to imaging are not intended to be specifically limited to the particular system shown in FIG. 1. Likewise, systems as described herein with respect to the application of localized energy for heating certain areas for thermal treatment are not intended to be specifically limited to the particular systems shown or described below.

One or more data acquisition or data collection steps as described herein in accordance with one or more embodiments may include acquiring, collecting, receiving, or otherwise obtaining data such as imaging data corresponding to an area of interest. By way of example, data acquisition or collection may include acquiring data via a data acquisition device, receiving data from an on-site or off-site data acquisition device or from another data collection, storage, or processing device. Similarly, data acquisition or data collection devices of a system in accordance with one or more embodiments of the present disclosure may include any device configured to acquire, collect, or otherwise obtain data, or to receive data from a data acquisition device within the system, an independent data acquisition device located on-site or off-site, or another data collection, storage, or processing device.

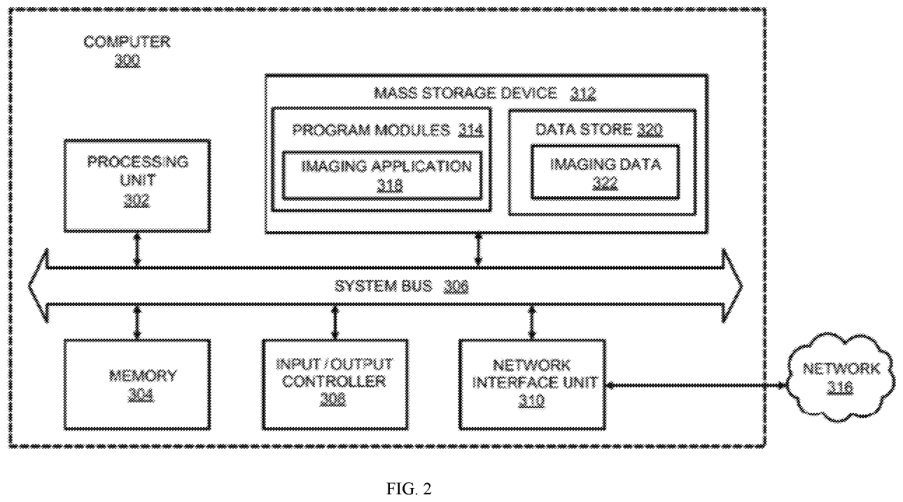

FIG. 2 is a computer architecture diagram showing a computing system capable of implementing aspects of the present disclosure in accordance with one or more embodiments described herein. A computer 300 may be configured to perform one or more specific steps of a method and/or specific functions for a system. The computer may be configured to perform one or more functions associated with embodiments illustrated in one or more of the figures herein. For example, the computer 300 may be configured to perform aspects described herein for implementing the pulse sequences shown and for various aspects of magnetic resonance imaging and related signal and temperature monitoring shown in the figures. It should be appreciated that the computer 300 may be implemented within a single computing device or a computing system formed with multiple connected computing devices. The computer 300 may be configured to perform various distributed computing tasks, in which processing and/or storage resources may be distributed among the multiple devices. The data acquisition and display computer 150 and/or operator console 110 of the system shown in FIG. 1, and the controller 212 and/or MRI electronics 210 of the system shown in FIG. 2, may include one or more components of the computer 300.

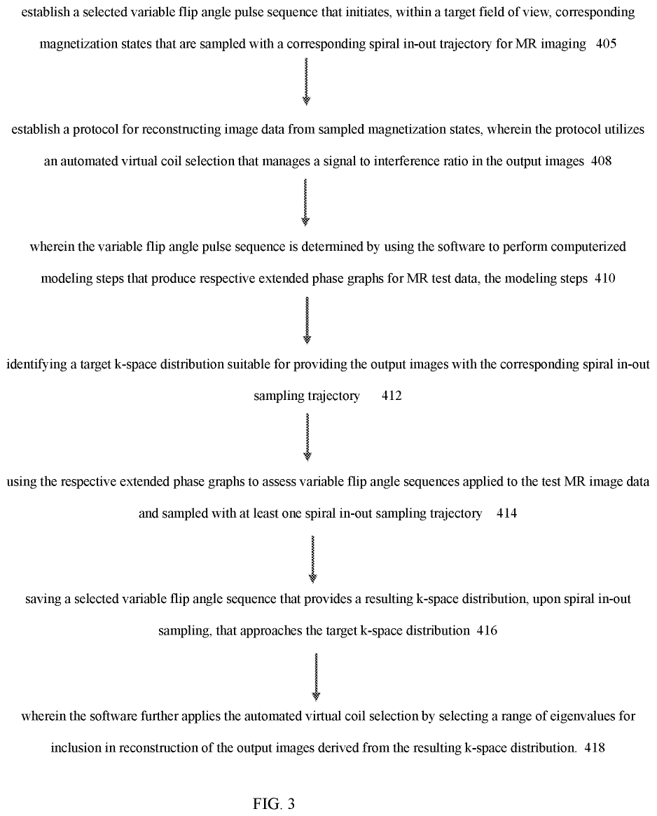

As shown, the computer 300 includes a processing unit 302 (“CPU”), a system memory 304, and a system bus 306 that couples the memory 304 to the CPU 302. The computer 300 further includes a mass storage device 312 for storing program modules 314. The program modules 314 may be operable to perform functions associated with one or more embodiments described herein. For example, when executed, the program modules can cause one or more medical imaging devices, localized energy producing devices, and/or computers to perform functions described herein for implementing the pulse sequence shown in FIG. 3, the method shown in FIG. 1, and for various aspects of magnetic resonance imaging and related signal and temperature monitoring and analysis shown in the figures herein. The program modules 314 may include an imaging application 318 for performing data acquisition and/or processing functions as described herein, for example to acquire and/or process image data corresponding to magnetic resonance imaging of an area of interest. The computer 300 can include a data store 320 for storing data that may include imaging-related data 322 such as acquired data from the implementation of magnetic resonance imaging pulse sequences in accordance with various embodiments of the present disclosure.

The mass storage device 312 is connected to the CPU 302 through a mass storage controller (not shown) connected to the bus 306. The mass storage device 312 and its associated computer-storage media provide non-volatile storage for the computer 300. Although the description of computer-storage media contained herein refers to a mass storage device, such as a hard disk, it should be appreciated by those skilled in the art that computer-storage media can be any available computer storage media that can be accessed by the computer 300.

By way of example and not limitation, computer storage media (also referred to herein as “computer-readable storage medium” or “computer-readable storage media”) may include volatile and non-volatile, removable and non-removable media implemented in any method or technology for storage of information such as computer-storage instructions, data structures, program modules, or other data. For example, computer storage media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer 300. “Computer storage media”, “computer-readable storage medium” or “computer-readable storage media” as described herein do not include transitory signals.

According to various embodiments, the computer 300 may operate in a networked environment using connections to other local or remote computers through a network 316 via a network interface unit 310 connected to the bus 306. The network interface unit 310 may facilitate connection of the computing device inputs and outputs to one or more suitable networks and/or connections such as a local area network (LAN), a wide area network (WAN), the Internet, a cellular network, a radio frequency (RF) network, a Bluetooth-enabled network, a Wi-Fi enabled network, a satellite-based network, or other wired and/or wireless networks for communication with external devices and/or systems.

The computer 300 may also include an input/output controller 308 for receiving and processing input from any of a number of input devices. Input devices may include one or more of keyboards, mice, stylus, touchscreens, microphones, audio capturing devices, and image/video capturing devices. An end user may utilize the input devices to interact with a user interface, for example a graphical user interface, for managing various functions performed by the computer 300. The input/output controller 308 may be configured to manage output to one or more display devices for displaying visually representations of data, such as display monitors/screens that are integral with other components of the computer 300 or are remote displays.

The bus 306 may enable the processing unit 302 to read code and/or data to/from the mass storage device 312 or other computer-storage media. The computer-storage media may represent apparatus in the form of storage elements that are implemented using any suitable technology, including but not limited to semiconductors, magnetic materials, optics, or the like. The computer-storage media may represent memory components, whether characterized as RAM, ROM, flash, or other types of technology. The computer storage media may also represent secondary storage, whether implemented as hard drives or otherwise. Hard drive implementations may be characterized as solid state, or may include rotating media storing magnetically-encoded information. The program modules 314, which include the imaging application 318, may include instructions that, when loaded into the processing unit 302 and executed, cause the computer 300 to provide functions associated with one or more embodiments illustrated in the figures. The program modules 314 may also provide various tools or techniques by which the computer 300 may participate within the overall systems or operating environments using the components, flows, and data structures discussed throughout this description.

The present disclosure sets forth to enhance image quality of free-breathing spiral cardiac cine imaging at 3T, with a particular focus on improving image contrast and reducing artifacts.

Example, Non-limiting Goal(s): This study aims to, among other things, demonstrate that optimizing both data acquisition and the reconstruction algorithm can enhance overall image quality by improving image contrast and reducing artifacts.

Example, Non-limiting Approach: A variable flip angle scheme based on extended phase graphs CEPG) simulation and a region-optimized virtual coils algorithm were developed.

Example, Non-limiting Results: The in vivo experiments demonstrate that, among other things, the proposed methods outperform the conventional acquisition and reconstruction methods at 8-fold under-sampling rate.

The present disclosure submits that reduced image contrast, along with interference artifacts can restrict the clinical use of free-breathing spiral cardiac cine imaging at 3T. The optimizations presented here offer significant potential to, among other things, broaden the clinical applicability of this technique at 3T.

### Methods

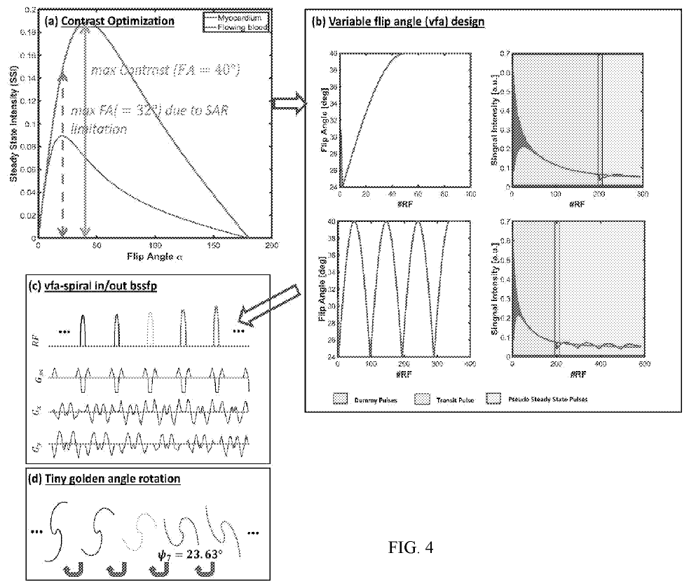

Variable flip angle scheme: The maximum allowable flip angle for one example acquisition parameters is 32°. However, the maximum blood-myocardium contrast is achieved at a flip angle of 40° (FIG. 4A). Increasing TR could allow for a higher flip angle but at the risk of introducing banding artifacts. To address this without increasing SAR, this disclosure designed a variable flip angle scheme. Specifically, this disclosure modified the extended-phase-graph (EPG)-based optimized flip angle scheme from Ref [B3] for bSSFP pulse sequences to simulate flip angles within a single measurement (a single measurement consists of fully sampled spiral interleaves). The resulting simulated flip angle curve closely resembles a sine wave. For ease of implementation in continuous dynamic acquisition, this disclosure approximated this curve with a sine wave, as suggested in Ref [B4].

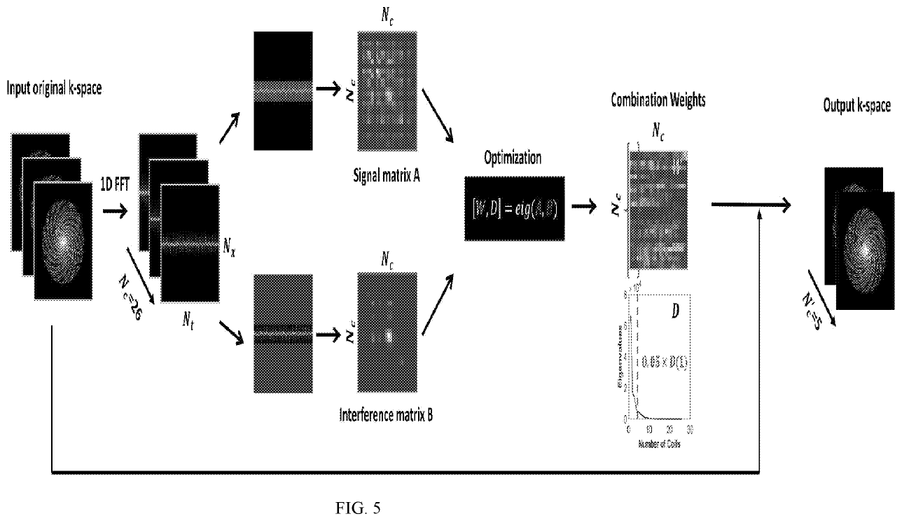

Region-optimized virtual coils algorithm: While conventional single value decomposition (SVD)-based coil compression improves computational efficiency, it does not adequately suppress artifacts. The ROVir algorithm [B5] was initially proposed to suppress unwanted signals by maximizing signal-intensity-ratio (SIR), with its application to dynamic radial cardiac imaging detailed in Ref [B6]. This disclosure adapted this approach to mitigate the swirl artifacts for the study. To automate virtual coil selection for optimal signal interference ratio (SIR), this disclosure retained those eigenvalues greater than 3% of the first eigenvalue (in descending order) (FIG. 5). The processed k-space data were then reconstructed using the L+S algorithm [B7].

Simulation & Volunteer Imaging: T1/T2 values of blood and myocardium in the simulation were set to 1932/2753 ms and 1471/47 ms, respectively. Imaging was performed on two healthy volunteers using a 3T Siemens Prisma scanner, with IRB approval and written informed consent. Imaging Parameters included: FOV=330×330 mm², TR/TE=4.4/2.2 ms, in-plane spatial resolution=1.5×1.5 mm, slice thickness=8 mm, and an under-sampling rate of 8. A dual-density spiral from 2.3× to 0.4× Nyquist was designed for the spiral in/out trajectories. Each measurement contains 96 spiral interleaves, with 20 measurements for dynamic imaging. A tiny golden angle increment of 23.63° was used to rotate the spiral interleaves.

For the flip angle scheme, the constant flip angle (CFA) was set to 32°. The variable flip angle started with a transient RF pulse at 31° and cycled from a low flip angle of 24° to a high flip angle of 40°, repeating across the 20 measurements with a period of 96 TRs.

### Embodiments

In one implementation, a computer implemented method of specifying a magnetic resonance (MR) imaging protocol to provide output images with high contrast sections and reduced artifacts includes using a computer having a processor in data communication with computer memory storing software that implements steps to establish a selected variable flip angle pulse sequence that initiates, within a target field of view, corresponding magnetization states that are sampled with a corresponding spiral in-out trajectory for MR imaging 405; and establish a protocol for reconstructing image data from sampled magnetization states, wherein the protocol utilizes an automated virtual coil selection that manages a signal to interference ratio in the output images 408. The variable flip angle pulse sequence is determined by using the software to perform computerized modeling steps that produce respective extended phase graphs for MR test data, the modeling steps comprising identifying a target k-space distribution suitable for providing the output images with the corresponding spiral in-out sampling trajectory 410; using the respective extended phase graphs to assess variable flip angle sequences applied to the test MR image data and sampled with at least one spiral in-out sampling trajectory 412; using the respective extended phase graphs to assess variable flip angle sequences applied to the test MR image data and sampled with at least one spiral in-out sampling trajectory 414; saving a selected variable flip angle sequence that provides a resulting k-space distribution, upon spiral in-out sampling, that approaches the target k-space distribution 416 and wherein the software further applies the automated virtual coil selection by selecting a range of eigenvalues for inclusion in reconstruction of the output images derived from the resulting k-space distribution 418.

In other implementations, the MR imaging protocol is a balanced steady state free precession (bSSFP) protocol.

In other implementations, the MR imaging protocol utilizes a magnetic field greater than 1.5 Tesla.

In other implementations, the MR imaging protocol utilizes a three Tesla (3T) magnetic field.

In other implementations, the automated virtual coil selection comprises a region optimized virtual coil (ROVir) algorithm.

In other implementations, the eigenvalues retained for the output images are each greater than 5 percent of a first eigenvalue.

In other implementations, the eigenvalues retained for the output images are each greater than 5 percent of a first eigenvalue and utilized in a descending order.

In other implementations, producing the extended phase graphs comprises providing the software with set-up parameters comprising the field of view (FOV)=330×330 mm², TR/TE=4.4/2.2 ms, in-plane spatial resolution=1.5×1.5 mm, slice thickness=8 mm, and an under-sampling rate of 8.

In other implementations, the corresponding spiral in-out sampling trajectory comprises a dual density spiral from 2.3 to 0.4 times the Nyquist rate.

In other implementations, the variable flip angle pulse sequence comprises a low flip angle of 24° to a high flip angle of 40°.

In other implementations, the corresponding spiral in-out sampling trajectory comprises a golden angle rotation of 23.63°.

In a computer implemented method of acquiring magnetic resonance (MR) image data of an area of interest of a subject with a selected spiral in-out sampling trajectory in conjunction with a selected variable flip angle pulse sequence according to Claim 1.

In other implementations, acquiring the output images from the resulting K-space data set further comprises steps of:
dividing the resulting K-space data set into a signal region and an interference region;
computing correlation matrices from the signal region and the interference region;
calculating a weights matrix through eigen-decomposition;
truncating the eigen-decomposition based on the eigenvalues exceeding 5 percent of the first eigenvalue;
applying the weights matrix to the k-space data set to form a processed k-space data set; and
preparing the output images from the processed k-space data set by applying a low rank plus sparse reconstruction algorithm.

As further description the disclosure includes:

FIG. 1. Overview of variable flip angle design and spiral in/out BSSFP pulse sequence. (a) Simulation of steady state signal intensity as a function of flip angles. (b) Optimized variable flip angle scheme for a single measurement based on EPG simulation, with sine modification across four measurements for ease of implementation in dynamic imaging as shown below. (c) Diagram of variable-flip-angle-based spiral in/out bSSFP pulse sequence. (d) Illustration of tiny golden angle rotation to mitigate eddy ~current-induced artifacts.
FIG. 2. Illustration of region-optimized virtual coils algorithm. The raw k-space data undergoes a 1D FFT, after which k-space is divided into two regions: the signal region and the interference region. Correlation matrices, A and B, are computed from these respective regions. The combination weights matrix, W, is calculated through eigen-decomposition and truncated based on eigenvalues exceeding 5% of the first eigenvalue. The final k-space output is obtained by applying the W to the raw k-space data.
FIG. 3. Comparison of reconstructed images at an under-sampling rate of R=8 from CFA and CFA acquisitions in basal and mid slices. Two representative temporal frames, end systole and end diastole, are shown, with a zoomed-in region (red box) to enhance visualization of details. FIG. 4, Quantitative analysis of CFA and VFA acquisitions in terms of CNR withm one heartbeat for basal and mid slices. CNR values were calculated between the left ventricle (LV) and myocardium. For statistical analysis in each image frame, 15 ROIs were randomly selected—5 each from LV, myocardium, and noise areas.
FIG. 4. Quantitative analysis of CFA and VFA acquisitions in terms of CNR within one heartbeat for basal and mid slices. CNR values were calculated between the left ventricle (LV) and myocardium. For statistical analysis in each image frame, 15 ROIs were randomly selected—5 each from LV, myocardium, and noise areas.
FIG. 5 Reconstructed free-breathing spiral cardiac cines from VFA acquisition using two different coil compression methods, Dynamic cardiac cine image of three representative slices. The calculated IR values for each temporal frame from top to bottom) are shown. The relative interference ratio (IR) also shown.

### REFERENCES

The following references are listed to provide attribution by reference number. Each of these references is incorporated by reference as if set forth fully herein. The following patents, applications and publications as listed below and throughout this document are hereby incorporated by reference in their entirety herein, and which are not admitted to be prior art with respect to the present invention by inclusion in this section.
1. Keltner JR, Roos MS, Brakeman PR, Budinger TF. Magnetohydrodynamics of blood flow. Magn. Reson. Med. 1990; 16:139-49.
2. Polson MJR, Barker AT, Gardiner S. The effect of rapid rise-time magnetic fields on the ECG of the rat. Clin. Phys. Physiol. Meas. 1982; 3:231-4.
3. Shetty AN. Suppression of Radiofrequency Interference in Cardiac Gated MRI: A Simple Design. Magn. Reson. Med. 1988; 8:84-8.
4. Damji AA, Snyder RE, Ellinger DC, Witkowski FX, Allen PS. RF interference suppression in a cardiac synchronization system operating in a high magnetic field NMR imaging system. Magn. Reson. Imaging. 1988; 6:637-40.
5. Firmin D, Keegan J. Navigator echoes in cardiac magnetic resonance. J. Cardiovasc. Magn. Reson. 2001; 3:183-93.
6. Jung B, Zaitsev M, Hennig J, Markl M. Navigator gated high temporal resolution tissue phase mapping of myocardial motion. Magn. Reson. Med. 2006; 55:937-42.
7. Peters DC, Nezafat R, Eggers H, Stehning C, Manning WJ. 2D free-breathing dual navigator-gated cardiac function validated against the 2D breath-hold acquisition. J. Magn. Reson. Imaging. 2008; 28:773-7.
8. Abd-Elmoniem KZ, Obele CC, Sibley CT, Matta JR, Pettigrew RI, Gharib AM. Free Breathing Single Navigator Gated Cine Cardiac Magnetic Resonance at 3 Tesla: Feasibility Study in Patients. J Comput Assist Tomogr. 2011; 35:382-6.
9. Moghari MH, Barthur A, Amaral ME, Geva T, Powell AJ. Free-breathing whole-heart 3D cine magnetic resonance imaging with prospective respiratory motion compensation. Magn. Reson. Med. [Internet]. 2017; 189:181-9. Available from: http://doi.wiley.com/10.1002/mrm.27021
10. Crowe ME, Larson AC, Zhang Q, Carr J, White RD, Li D, et al. Automated rectilinear self-gated cardiac cine imaging. Magn. Reson. Med. 2004; 52:782-8.
11. Buehrer M, Curcic J, Boesiger P, Kozerke S. Prospective self-gating for simultaneous compensation of cardiac and respiratory motion. Magn. Reson. Med. 2008; 60:683-90.
12. Uribe S, Muthurangu V, Boubertakh R, Schaeffter T, Razavi R, Hill DLG, et al. Whole-heart cine MRI using real-time respiratory self-gating. Magn. Reson. Med. 2007; 57:606-13.
13. Uribe S, Beerbaum P, Sørensen TS, Rasmusson A, Razavi R, Schaeffter T. Four-dimensional (4D) flow of the whole heart and great vessels using real-time respiratory self-gating. Magn. Reson. Med. 2009; 62:984-92.
14. Feng X, Salerno M, Kramer CM, Meyer CH. Non-Cartesian balanced steady-state free precession pulse sequences for real-time cardiac MRI. Magn. Reson. Med. 2016; 75:1546-55.
15. Stehning C, B? ?rnert P, Nehrke K, Eggers H, Stuber M. Free-breathing whole-heart coronary MRA with 3D radial SSFP and self-navigated image reconstruction. Magn. Reson. Med. 2005; 54:476-80.
16. Lai P, Bi X, Jerecic R, Li D. A Dual-Projection Respiratory Self-Gating Technique for Whole-Heart Coronary MRA. Magn. Reson. Med. 2009; 62:731-8.
17. Lai P, Bi X, Jerecic R, Li D. A respiratory self-gating technique with 3D-translation compensation for free-breathing whole-heart coronary MRA. Magn. Reson. Med. 2009; 62:731-8.
18. Usman M, Atkinson D, Odille F, Kolbitsch C, Vaillant G, Schaeffter T, et al. Motion corrected compressed sensing for free-breathing dynamic cardiac MRI. Magn. Reson. Med. 2013; 70:504-16.
19. Pang J, Bhat H, Sharif B, Fan Z, Thomson LEJ, Labounty T, et al. Whole-heart coronary MRA with 100% respiratory gating efficiency: Self-navigated three-dimensional retrospective image-based motion correction (TRIM). Magn. Reson. Med. 2014; 71:67-74.
20. Usman M, Ruijsink B, Nazir MS, Cruz G, Prieto C. Free breathing whole-heart 3D CINE MRI with self-gated Cartesian trajectory. Magn. Reson. Imaging. The Authors; 2017; 38:129-37.
21. Liu J, Spincemaille P, Codella NCF, Nguyen TD, Prince MR, Wang Y. Respiratory and cardiac self-gated free-breathing cardiac CINE imaging with multiecho 3D hybrid radial SSFP acquisition. Magn. Reson. Med. 2010; 63:1230-7.
22. Pang J, Sharif B, Fan Z, Bi X, Arsanjani R, Berman DS, et al. ECG and navigator-free four-dimensional whole-heart coronary MRA for simultaneous visualization of cardiac anatomy and function. Magn. Reson. Med. 2014; 72:1208-17.
23. Larson AC, White RD, Laub G, McVeigh ER, Li D, Simonetti OP. Self-Gated Cardiac Cine MRI. Magn. Reson. Med. 2004; 51:93-102.
24. Han F, Rapacchi S, Hu P. Prospective cardiac motion self-gating. Quant Imaging Med Surg. 2017; 2:215-26.
25. Bonanno G, Hays AG, Weiss RG, Schär M. Self-gated golden angle spiral cine MRI for coronary endothelial function assessment. Magn. Reson. Med. 2017; 00:1-11.
26. Odille F, Uribe S, Batchelor PG, Prieto C, Schaeffter T, Atkinson D. Model-based reconstruction for cardiac cine MRI without ECG or breath holding. Magn. Reson. Med. 2010; 63:1247-57.
27. Brau ACS, Brittain JH. Generalized self-navigated motion detection technique: Preliminary investigation in abdominal imaging. Magn. Reson. Med. 2006; 55:263-70.
28. Larson AC, Kellman P, Arai A, Hirsch GA, McVeigh E, Li D, et al. Preliminary investigation of respiratory self-gating for free-breathing segmented cine MRI. Magn. Reson. Med. 2005; 53:159-68.
[B1] Feng X, Salerno M, Kramer CM, Meyer CH. Non-Cartesian balanced steady-state free precession pulse sequences for real-time cardiac MRI. Magn Reson Med. 2016 April, 7S(4): 1546-55.
[B2] Wang Z, Feng X, Salerno M, Kramer CM, Meyer CH. Dynamic cardiac MRI with high spatiotemporal resolution using accelerated spiral-out and spiral-in/out bSSFP pulse sequences at 1.5 T. MAGMA. 2023 December 36(6):85 7-867.
[B3] Rahbek S, Schakel T, Mahmood F, Madsen KH, Phippens MEP, Hanson LG. Optimized flip angle schemes for the split acquisition of fast spin-echo signals (SPLICE) sequence and application to diffusion-weighted imaging. Magn Reson Med. 2023 April 89(4): 1469-1480.
[B4] Hennig J, Weigel M, Scheffler K. Muluecho sequences with variable refocusing flip angles: optimization of signal behavior using smooth transitions betwoen pseudo steady states (TRAPS). Magn Reson Med 2003March 49(3):527-38.
[B5] Kim D, Cauley SF, Nayak KS, Leahy RM, Haldar JP. Region-optimized virtual (ROVir) coils: Localization and/or suppression of spatial regions using sensor-domain beamforming. Magn Reson Med. 2021 July 86(1): 197-212.
[B6] Tut M, Ryu K, Li ZT, & Vasanawala S. Deep Learning Reconstruction for Free-Breathing Radial Cine Imaging. 2024. ISMRM #0043.
[B7] Otazo R, Candes E, Sodickson DK. Low-rank plus sparse matrix decomposition for accelerated dynamic MRI with separation of background and dynamic components. Magn Reson Med. 2015 March, 73(3):1125-36.
[B8] Brian Hargreaves and Karla Miller ISMRM 2013: Educational E-Poster #3718].

The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. The patentable scope of certain embodiments of the disclosed technology is indicated by the appended claims, rather than the foregoing description.

1. A computer implemented method of specifying a magnetic resonance (MR) imaging protocol to provide output images with high contrast sections and reduced artifacts, the method comprising:
using a computer having a processor in data communication with computer memory storing software that implements steps to:
establish a selected variable flip angle pulse sequence that initiates, within a target field of view, corresponding magnetization states that are sampled with a corresponding spiral in-out trajectory for MR imaging; and
establish a protocol for reconstructing image data from sampled magnetization states, wherein the protocol utilizes an automated virtual coil selection that manages a signal to interference ratio in the output images;
wherein the variable flip angle pulse sequence is determined by using the software to perform computerized modeling steps that produce respective extended phase graphs for MR test data, the modeling steps comprising:
identifying a target k-space distribution suitable for providing the output images with the corresponding spiral in-out sampling trajectory;
using the respective extended phase graphs to assess variable flip angle sequences applied to the test MR image data and sampled with at least one spiral in-out sampling trajectory;
saving a selected variable flip angle sequence that provides a resulting k-space distribution, upon spiral in-out sampling, that approaches the target k-space distribution, and
wherein the software further applies the automated virtual coil selection by selecting a range of eigenvalues for inclusion in reconstruction of the output images derived from the resulting k-space distribution.

2. The computer implemented method of claim 1, wherein the MR imaging protocol is a balanced steady state free precession (bSSFP) protocol.

3. The computer implemented method of claim 2, wherein the MR imaging protocol utilizes a magnetic field greater than 1.5 Tesla.

4. The computer implemented method of claim 3, wherein the MR imaging protocol utilizes a three Tesla (3T) magnetic field.

5. The computer implemented method of claim 1, wherein the automated virtual coil selection comprises a region optimized virtual coil (ROVir) algorithm.

6. The computer-implemented method of claim 1, wherein the eigenvalues retained for the output images are each greater than 5 percent of a first eigenvalue.

7. The computer implemented method of claim 6, wherein the eigenvalues retained for the output images are each greater than 5 percent of a first eigenvalue and utilized in a descending order.

8. The computer implemented method of claim 1, wherein producing the extended phase graphs comprises providing the software with set-up parameters comprising the field of view (FOV)=330×330 mm², TR/TE=4.4/2.2 ms, in-plane spatial resolution=1.5×1.5 mm, slice thickness=8 mm, and an under-sampling rate of 8.

9. The computer implemented method of claim 1, wherein the corresponding spiral in-out sampling trajectory comprises a dual density spiral from 2.3 to 0.4 times the Nyquist rate.

10. The computer implemented method of claim 1, wherein the variable flip angle pulse sequence comprises a low flip angle of 24° to a high flip angle of 40°.

11. The computer implemented method of claim 1, wherein the corresponding spiral in-out sampling trajectory comprises a golden angle rotation of 23.63°.

12. A computer implemented method of acquiring magnetic resonance (MR) image data of an area of interest of a subject with a selected spiral in-out sampling trajectory in conjunction with a selected variable flip angle pulse sequence according to claim 1.

13. The computer implemented method of claim 12, wherein acquiring the output images from the resulting K-space data set further comprises steps of:
dividing the resulting K-space data set into a signal region and an interference region;
computing correlation matrices from the signal region and the interference region;
calculating a weights matrix through eigen-decomposition;
truncating the eigen-decomposition based on the eigenvalues exceeding 5 percent of the first eigenvalue;
applying the weights matrix to the k-space data set to form a processed k-space data set; and
preparing the output images from the processed k-space data set by applying a low rank plus sparse reconstruction algorithm.
